(19) United States
(12) Patent Application Publication
(10) Pub. No.: US 2004/0101484 A1
(43) Pub. Date: May 27, 2004

(54) Nasal delivery of apomorphine in combination with glycol derivatives

(76) Inventors: Charanjit R. Behl, Hauppauge, NY (US); Vincent D. Romeo, Massapequa Park, NY (US); Raja G. Achari, Millington, NJ (US); Shamin Ahmed, Central Islip, NY (US); Jorge C. Demeireles, Syosset, NY (US); Tianquing Liu, Central Islip, NY (US); Anthony P. Sileno, Brookhaven Hamlet, NY (US)

Correspondence Address: Joshua King, GRAYBEAL JACKSON HALEY LLP, Suite 350, 155 - 108th Avenue NE, Bellevue, WA, 98004-5901, US

(21) Appl. No.: 10/701,956

(22) Filed: Nov. 5, 2003

Related U.S. Application Data
(63) Continuation of application No. 09/882,746, filed on Jun. 11, 2001.
Application No. 09/882,746 is a continuation of application No. 09/665,500, filed on Sep. 19, 2000, now abandoned.
Application No. 09/665,500 is a continuation-in-part of application No. 09/334,304, filed on Jun. 16, 1999, now Pat. No. 6,436,950.
(60) Provisional application No. 60/096,545, filed on Aug. 14, 1998.

(51) Int. Cl. A61L 9/04; A61K 9/14

(52) U.S. Cl. 424/46; 514/295

(57) ABSTRACT

Intranasal delivery compositions and methods for the delivery of dopamine receptor agonists are provided which are effective for the treatment14 of sexual dysfunction in a mammal without causing substantial intolerable adverse side effects to the mammal, in particular adverse nasal effects. Nasally administered compositions for treating sexual dysfunction in a mammal are also provided which include a therapeutically effective amount of a dopamine receptor agonist which has been dispersed in a system to improve its solubility and/or stability.

### CROSS REFERENCE TO RELATED APPLICATIONS

This application is a Continuing-in-Part Application of co-pending U.S. application Ser. No. 09/334,304, filed on Jun. 16, 1999

### BACKGROUND OF THE INVENTION

The present invention relates generally to intranasal delivery methods and dosage forms of apomorphine. More particularly, methods and dosage forms for the safe and reliable intranasal delivery of apomorphine with minimal adverse nasal effects to treat sexual dysfunction, including erectile dysfunction, in a mammal are provided.

Apomorphine is a potent dopamine receptor agonist which has a variety of uses. For example, it has been effectively used as an adjunctive medication in the treatment of Parkinson's disease which is complicated by motor fluctuations (T. van Laar et al., Arch. Neurol., 49: 482-484 (1992)). In particular, apomorphine has been used for relieving “off-period” symptoms in Parkinson patients with such response fluctuations. In the study by van Laar et al., the intranasally applied apomorphine used to achieve the results reportedly included an aqueous solution of apomorphine hydrochloride (HCL) at a concentration of 10 mg/ml. This formulation is also used for parenteral application and is published in different Pharmacopeia's.

Also, U.S. Pat. No. 5,756,483 issued to Merkus (hereinafter “the '483 patent”) which is hereby incorporated by reference, discloses the intranasal delivery of a variety of compositions, including apomorphine in combination with a cyclodextrin and/or a polysaccharide and/or a sugar alcohol for treating Parkinson's disease. The '483 patent, however, discloses very narrow dosage ranges of 0.1 to 2 mg of apomorphine per nostril which is specifically tailored for the treatment of the “off-period” symptoms of Parkinson's disease.

Further, U.S. Pat. No. 5,770,606 issued to El-Rashidy et al. (hereinafter “the '606 patent”), which is hereby incorporated by reference, discloses the delivery of apomorphine in a sublingual dosage unit for alleviating psychogenic impotence or erectile dysfunction with no substantial undesirable side effects. The '606 patent further includes results from a study conducted by the inventors on the effect of apomorphine delivered intranasally on erectile dysfunction. The study suggested that intranasal delivery of apomorphine at concentrations of 2.5 mg to 3.5 mg was effective for eliciting an erection in patients suffering from erectile dysfunction, however, since the study participants suffered extensive and serious side effects including hypotension, nausea, vomiting, impaired vision, diaphoresis and ashen coloring, it was concluded that intranasal delivery of apomorphine to treat erectile dysfunction was insufficiently safe and reliable to be a viable commercial product.

Accordingly, it is one of the purposes of this invention, among others, to provide a safe and reliable intranasal delivery system for apomorphine that ensures delivery of therapeutic amounts of the drug into the bloodstream which is fast acting, easily administered and causes no substantial adverse side effects, in particular adverse nasal side effects.

### SUMMARY OF THE INVENTION

It has now been discovered that this and other purposes can be achieved by the present invention, which provides for a method for ameliorating sexual dysfunction, including male and female erectile dysfunction, in a mammal. Ameliorating sexual dysfunction, for the purposes of this invention, includes reducing or eliminating the incidence or severity of the sexual dysfunction. This method includes the nasal administration of a dopamine receptor agonist to the mammal before, during or after sexual activity in an amount sufficient to ameliorate the sexual dysfunction, or in an amount sufficient to induce an erection in male or female erectile tissue, without inducing substantial intolerable side effects, in particular adverse nasal effects, in the mammal. Preferably, the dopamine receptor agonist is apomorphine with glycol derivatives. It has been unexpectedly found that glycol derivatives allow nasally administered apomorphine to be more tolerable due to an inhibition/elimination of nasal adverse effects.

The present invention also provides for a pharmaceutical composition for treating male and female sexual dysfunction, including male and female erectile dysfunction, in a mammal without causing substantial intolerable adverse side effects, in particular adverse nasal side effects, that include a therapeutically effective amount of a dopamine receptor agonist in combination with a nasal delivery system and glycol derivatives. Preferably, the dopamine receptor agonist is selected from the group consisting of apomorphine, chemically modified equivalents and pharmaceutical salts thereof; and further preferably, the dopamine receptor agonist is apomorphine. The chemically modified equivalents of apomorphine preferably include a pro-drug. Further, it is preferable that apomorphine is dispersed in an aqueous or non-aqueous formulation. Preferably the glycol derivative is propylene glycol.

In addition, the nasal delivery system of the pharmaceutical composition can include a buffer to maintain the pH of the dopamine receptor agonist, a pharmaceutically acceptable thickening agent and a humectant. The pharmaceutical composition can further include one or more pharmaceutical excipients and even further include a pharmaceutically acceptable preservative.

The buffer of the nasal delivery system can be selected from the group including acetate, citrate, prolamine, carbonate and phosphate buffers.

The thickening agent of the nasal delivery system can be selected from the group including methyl cellulose, xanthan gum, carboxymethyl cellulose, hydroxypropyl cellulose, carbomer, polyvinyl alcohol, alginates, acacia, chitosans and combinations thereof.

The humectant of the nasal delivery system can be selected from the group including sorbitol, glycerol, mineral oil, vegetable oil and combinations thereof.

The present invention also provides a method of treating sexual dysfunction in mammals including nasally administering a pharmaceutical composition which includes a therapeutically effective amount of a dopamine receptor agonist in 10 combination with a nasal delivery system wherein the pharmaceutical composition does not cause substantial intolerable adverse side effects in the mammal, in particular adverse nasal side effects. The sexual dysfunction to be treated by the present invention includes erectile dysfunction. Preferably, the dopamine receptor agonist is apomorphine with glycol derivatives. Preferably, the glycol derivative is propylene glycol.

The present invention also provides for a nasally administered pharmaceutical composition that includes a therapeutically effective amount of a dopamine receptor agonist dispersed in a buffer to maintain its pH, a glycol derivative, a pharmaceutically acceptable thickening agent and a humectant, wherein said nasally administered pharmaceutical composition does not cause substantial intolerable adverse side effects, in particular adverse nasal effects, when administered to a mammal. The dopamine receptor agonist of the nasally administered pharmaceutical composition is selected from the group including apomorphine, chemically modified equivalents and pharmaceutical salts thereof. It is further preferable that chemically modified equivalents of apomorphine include a pro-drug.

The present invention also provides for a method of treating male or female sexual dysfunction, including impotence and male or female erectile dysfunction, in a human in need of such treatment. The method includes administering to a nasal membrane of the human an effective amount of a nasally administered pharmaceutical composition including a therapeutically effective amount of a dopamine receptor agonist dispersed in a buffer to maintain its pH, a glycol derivative, a pharmaceutically acceptable thickening agent and a humectant, wherein the nasally administered pharmaceutical composition does not cause substantial intolerable adverse side effects, in particular adverse nasal effects, when administered to the human.

Another preferred method of the present invention provides for treating male or female sexual dysfunction, including male and female erectile dysfunction, in a mammal without causing substantial intolerable adverse side effects, in particular adverse nasal side effects. This method includes administering into a nasal cavity of the mammal a therapeutically effective dosage of a dopamine receptor agonist in combination with a nasal delivery system. The nasal delivery system includes a glycol derivative, a pharmaceutically acceptable buffer, a thickening agent and a humectant. The dopamine receptor agonist is selected from the group including apomorphine, chemically modified equivalents and pharmaceutical salts thereof. Preferably, chemically modified equivalents of apomorphine include a pro-drug.

Another preferred method of the present invention is a method for administering a therapeutically effective amount of a dopamine receptor agonist to a mammal through a nasal membrane without causing substantial intolerable adverse side effects, in particular adverse nasal effects. This method includes delivering to the nasal membrane of a mammal a dopamine receptor agonist dispersed in a nasal delivery system which includes a glycol derivative, a pharmaceutically acceptable buffer, a thickening agent and a humectant. Preferably, the dopamine receptor agonist is effective for the treatment of sexual dysfunction, including male and female erectile dysfunction, in a mammal.

The present invention also provides for an intranasal dosage unit for treating male and female sexual dysfunction, including impotency and erectile dysfunction in a mammal, which does not cause substantial intolerable adverse side effects, especially adverse nasal effects. The dosage unit includes an effective amount of a dopamine receptor agonist in combination with a glycol derivative with an intranasal carrier. The intranasal carrier includes a buffer. The buffer pH is selected to enhance absorption of the dopamine receptor agonist and to produce an erection within about 60 minutes of administering the dosage unit to a nasal mucosa of the mammal. Preferably, an erection is produced within about 45 minutes, more preferably within about 30 minutes, most preferably within about 15 minutes, and even further preferably in less than about 15 minutes.

The intranasal carrier of the intranasal dosage unit is preferably an aqueous solution. Further, the aqueous solution can be selected from the group including aqueous gels, aqueous suspensions, aqueous liposomal dispersions, aqueous emulsions, aqueous microemulsions and combinations thereof.

Alternatively, the intranasal carrier of the intranasal dosage unit is a nonaqueous solution. The non-aqueous solution can be selected from a group including non-aqueous gels, non-aqueous suspensions, non-aqueous liposomal dispersions, non-aqueous emulsions and non-aqueous microemulsions and combinations thereof.

The intranasal carrier of the intranasal dosage unit can also be a combination of an aqueous solution and a non-aqueous solution.

Alternatively, the carrier of the intranasal dosage unit is a powder formulation. The powder formulation can be selected from a group including simple powder mixtures, powder microspheres, coated powder microspheres, liposomal dispersions and combinations thereof. Preferably, the powder formulation is powder microspheres. The powder microspheres are preferably formed from various polysaccharides and celluloses selected from the group including starch, methylcellulose, xanthan gum, carboxymethylcellulose, hydroxypropyl cellulose, carbomer, alginate polyvinyl alcohol, acacia, chitosans and combinations thereof.

The intranasal dosage unit can also include an excipient having bio-adhesive properties. Preferably, the buffer of the intranasal dosage unit is selected to have a pH of from about 3 to about 10 and more preferably from about 3.5 to 7.0.

Preferably, the intranasal dosage unit includes a humectant. A humectant can be selected from the group consisting of soothing agents, membrane conditioners, sweeteners and combinations thereof.

The present invention also provides for a nasally administered pharmaceutical composition for treating sexual dysfunction in a mammal including a therapeutically effective amount of a dopamine receptor agonist which has been dispersed in a system to improve its solubility and preferably a glycol derivative. Preferred glycol derivatives include propylene glycol and polyethylene glycol. Sexual dysfunction |includes male and female erectile dysfunction. The dopamine receptor agonist of this composition is selected from the group consisting of apomorphine, chemically modified equivalents and pharmaceutical salts thereof. The system of this composition includes one of the following or combinations thereof: a sugar alcohol; glycerin; propylene glycol and glycerin; polyethylene glycol 400; ascorbic acid and water; sodium ascorbate and water; or sodium metabisulfite and water. Preferred sugar alcohols include mannitol and xylitol.

The present invention also provides for a nasally administered pharmaceutical composition for treating sexual dysfunction in a mammal including a therapeutically effective amount of a dopamine receptor agonist which has been dispersed in a system to improve its stability. Sexual dysfunction includes male and female erectile dysfunction. The dopamine receptor agonist of this composition is selected from the group consisting of apomorphine, chemically modified equivalents and pharmaceutical salts thereof. The composition preferably includes a glycol derivative, in particular propylene glycol or polyethylene glycol. The system of this composition includes one of the following or combinations thereof: a sugar alcohol; glycerin; propylene glycol and glycerin; polyethylene glycol 400; ascorbic acid and water; sodium ascorbate and water; or sodium metabisulfite and water. Preferred sugar alcohols include mannitol and xylitol.

These and other advantages of the present invention will be appreciated from the detailed description and examples which are set forth herein. The detailed description and examples enhance the understanding of the invention, but are not intended to limit the scope of the invention.

### DETAILED DESCRIPTION OF THE INVENTION

The present invention is directed to compositions and methods for treating sexual dysfunction in a mammal. In particular, a method is provided for ameliorating male and female sexual dysfunction in a mammal by nasally administering to the mammal a therapeutically effective amount of a dopamine receptor agonist before, during or after sexual activity without causing substantial adverse side effects, in particular adverse nasal effects, in the mammal. “Ameliorating sexual dysfunction,” for the purposes of this invention, includes reducing or eliminating the incidence or severity of the sexual dysfunction. The method includes treating male and female erectile dysfunction in a mammal by nasally administering a therapuetically effective amount of a dopamine agonist which is sufficient to induce an erection without causing substantial adverse side effects. The composition preferably. includes glycol derivatives, in particular propylene glycol or polyethlyene glycol. It has now been unexpectedly found that glycol derivatives allow for nasally-administered apomorphine to be more tolerable due to an inhibition and/or elimination of adverse nasal effects.

For the purposes of the present invention, the phrase “sexual dysfunction” is intended to encompass “erectile dysfunction” in males and females, as well as lack of sexual desire in males and females. For purposes of the present invention, the phrase “erectile dysfunction” is intended to encompass certain medically related symptoms resulting in the inability of a male to perform sexually, including penile dysfunction, as well as male impotence. As used herein, the term “impotence” is intended to mean the inability of a male to achieve and/or sustain a penile erection sufficient for vaginal penetration and intercourse. “Erectile dysfunction” is also intended to encompass female erectile tissue dysfunction, including erectile tissue dysfunction which diminishes sexual stimulation. Additionally, for the purposes of the present invention, the phrase “sexual dysfunction” is intended to encompass any one of the following components, or any combination of the following components: decreased sexual desire; decreased sexual arousal; dyspareunia; and/or persistent difficulty in achieving or inability to achieve orgasm.

As used herein, a “dopamine receptor agonist” is intended to encompass those members of the dopamine receptor agonist family which are able to ameliorate sexual dysfunction, including male and female erectile dysfunction when administered to a mammal, as well as lack of sexual desire in humans. Apomorphine is an example of such a composition. Thus, the present invention is intended to encompass apomorphine and its functional equivalents including pharmaceutical salts and chemically modified equivalents thereof, including for example pro-drug forms of apomorphine. Apomorphine can be represented by the formula:

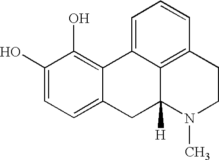

and in the present invention can exist in a free base form or as an acid addition salt. For the purposes of the present invention, apomorphine hydrochloride is preferred; however, other pharmacologically acceptable moieties thereof can be utilized as well. The term “apomorphine” as used herein includes the free base form of this compound as well as, pharmacologically acceptable acid addition salts thereof. In addition to the hydrochloride salt of apomorphine, other pharmacologically acceptable acid addition salts of apomorphine include the hydrobromide, the hydroiodide, the bisulfate, the phosphate, the acid phosphate, etc.

For the purposes of the present invention, apomorphine or a similarly acting dopamine receptor agonist is administered nasally in an amount sufficient to excite cells in the mid-brain region of the patient but without substantial adverse side effects. This cell excitation is believed to be part of a cascade of stimulation that is likely to include neurotransmission with serotonin and oxytocin. Because dopamine receptors agonists act directly on regions of the mid-brain, the present invention also contemplates the use of such agonists for improving the sexual desire in both male and female mammals, as well as the amelioration of erectile dysfunction in males and females as set forth above. The compositions and methods of the present invention include increasing the sexual desire in both males and females.

The dopamine receptors in the mid-brain region of a patient can be stimulated to a degree sufficient to cause an erection by the nasal administration of apomorphine so as to maintain an adequate plasma concentration of apomorphine. The amount of apomorphine nasally administered is an amount sufficient to ameliorate sexual dysfunction, including the amount sufficient to cause an erection, but is low enough not to cause substantial intolerable adverse side effects. In particular, adverse nasal effects are inhibited and/or eliminated by the compositions and methods of the present invention. Nasal adverse effects include, but are not limited to, sneezing; nasal burning; nasal itching; nasal pain; unusual taste, smell/odor; and congestion. As used herein, “substantial intolerable adverse side effects” include those effects caused by either the delivery system or the dopamine receptor agonist which are incompatible with the health of the user or which are so unpleasant as to discourage the continued use of the composition. Such effects include, for example, hypotension, nausea, vomiting, impaired vision, diaphoresis and ashen coloring.

Apomorphine is nasally administered about 30 to about 45 minutes prior to sexual activity, preferably about 15 to about 20 minutes prior to sexual activity, and more preferably less than 15 minutes prior to sexual activity.

The compositions according to the present invention can be administered, for example, as a nasal spray, nasal drop, suspension, gel, ointment, cream or powder. The administration of a composition can also include using a nasal tampon or a nasal sponge containing a composition of the present invention.

The dopamine receptor agonist can also be brought into a viscous basis via systems conventionally used, for example, natural gums, methylcellulose and derivatives, acrylic polymers (carbopol) and vinyl polymers (polyvinylpyrrolidone). In the present compositions, many other excipients known in the art can be added such as preservatives, surfactants, co-solvents, adhesives, antioxidants, buffers, viscosity enhancing agents and agents to adjust the pH and the osmolarity.

The amount of dopamine receptor agonist administered to a patient will vary according to the delivery system used, and the age and weight of the patient. There are two critical parameters for selecting the appropriate dosage levels of the dopamine receptor agonist. First, the dosage level must be effective for achieving an erection in the patient and second, the dosage level must not cause substantial intolerable adverse side effects to the patient.

The onset of substantial intolerable adverse side effects, for example, nausea and/or vomiting, can be obviated or delayed by nasally delivering a dopamine receptor agonist at a controlled dissolution rate so as to provide circulating serum levels and mid-brain tissue levels of the dopamine receptor agonist sufficient for an erection and without inducing nausea and/or vomiting. When it is necessary to administer higher doses of a dopamine receptor agonist, for example, doses above about 2 mg, the likelihood of a substantial intolerable adverse side effect onset can be reduced by concurrently administering a ganglionic agent capable of inhibiting the ganglionic response, for example, nicotine or lobeline sulfate.

Other antiemetic agents that can be used in accordance with the present invention include metoclopramide; phenothiazines such as chlorpromazine, prochlorperazine, pipamazine, thiethylperazine and oxypendyl hydrochloride; serotonin (5-hydroxytryptamine or 5-HT) agonists such as domperidone, odansetron and histamine antagonists including buclizine hydrochloride, cyclizine hydrochloride and dimenhydrinate; parasympathetic depressants such as scopolamine; metopimazine; trimethobenzamide; benzquinamine hydrochloride; and diphenidol hydrochloride.

As set forth previously, the nasal delivery systems that can be used with the present invention can take various forms including aqueous solutions, non-aqueous solutions and combinations thereof. Aqueous solutions include, for example, aqueous gels, aqueous suspensions, aqueous liposomal dispersions, aqueous emulsions, aqueous microemulsions and combinations thereof. Non-aqueous solutions include, for example, non-aqueous gels, non-aqueous suspensions, non-aqueous liposomal dispersions, non-aqueous emulsions, non-aqueous microemulsions and combinations thereof.

The various forms of the delivery system set forth above can include a buffer to maintain the pH of the dopamine receptor agonist, a pharmaceutically acceptable thickening agent and a humectant. Desirably, the pH of the buffer is selected to maintain the dopamine receptor agonist in a non-ionized form. In particular, the pH of the buffer is selected to optimize the absorption of the dopamine receptor agonist across the nasal mucosa. The particular pH of the buffer, of course, can vary depending upon the particular nasal delivery formulation as well as the specific dopamine receptor agonist composition selected. Buffers that are suitable for use in the present invention include acetate, citrate, prolamine, carbonate and phosphate buffers.

With respect to the non-aqueous formulations set forth above, suitable forms of buffering agents can be selected such that when the formulation is delivered into the nasal cavity of a mammal, selected pH ranges are achieved therein upon contact with, e.g., a nasal mucosa.

In the present invention, the pH of the. compositions should be maintained from about 3.0 to about 10.0. Compositions having a pH of less than about 3.0 or greater than about 10.0 can increase the risk of irritating the nasal mucosa of a recipient. Further, it is preferable that the pH of the compositions be maintained from a lower limit of about 3.0, 4.0 or 5.0 to an upper limit of about 4.0, 5.0, 6.0 or 7.0.

The viscosity of the compositions of the present invention can be maintained at a desired level using a pharmaceutically acceptable thickening agent. Thickening agents that can be used in accordance with the present invention include methyl cellulose, xanthan gum, carboxymethyl cellulose, hydroxypropyl cellulose, carbomer, polyvinyl alcohol, alginates, acacia, chitosans and combinations thereof. The concentration of the thickening agent will depend upon the agent selected and the viscosity desired. Such agents can also be used in a powder formulation discussed above.

The compositions of the present invention can also include a humectant to reduce or prevent drying of the mucus membrane and to prevent irritation thereof. Suitable humectants that can be used in the present invention include sorbitol, mineral oil, vegetable oil and glycerol; soothing agents; membrane conditioners; sweeteners; and combinations thereof. The concentration of the humectant in the present compositions will vary depending upon the agent selected.

In the present invention, other optional ingredients can also be incorporated into the nasal delivery system provided that they do not interfere with the action of the dopamine receptor agonist or significantly decrease the absorption of the dopamine receptor agonist across the nasal mucosa. Such ingredients include pharmaceutically acceptable excipients and preservatives.

To extend shelf life, preservatives can be added to the present compositions. Suitable preservatives that can be used with the present compositions include benzyl alcohol, parabens, thimerosal, chlorobutanol and benzalkonium chloride and preferably benzalkonium chloride is used. Typically, the preservative will be present in a composition in a concentration of up to about 2% by weight. The exact concentration of the preservative, however, will vary depending upon the intended use and can be easily ascertained by one skilled in the art.

The present invention provides for the compositions as described above which are administered nasally to a mammal to treat sexual dysfunction, including erectile dysfunction. For purposes of the present invention, “administered nasally” or “nasal administration” is intended to mean that the dopamine receptor agonists are combined with a suitable delivery system for absorption across the nasal mucosa of a mammal, preferably, a human.

A preferred embodiment of the present invention provides for a nasally administered pharmaceutical composition that includes a therapeutically effective amount of a dopamine receptor agonist dispersed in a buffer to maintain the pH of the agonist, a pharmaceutically acceptable thickening agent and a humectant. As used herein, “therapeutically effective amount” means a unit dosage of the present dopamine receptor agonist which is able to be combined with a pharmaceutically acceptable nasal delivery system and absorbed through the nasal mucosa of a mammal to produce an erection in about I hour, preferably in about 45 minutes, more preferably in about 30 minutes, and most preferably in 15 minutes or less which renders the intended physiological effect which is to induce an erection in a mammal, including a penile erection, in a mammal with erectile dysfunction including, penile erectile dysfunction, without causing substantial intolerable adverse side effects to the mammal. Preferably, the dopamine receptor agonist is selected from a group including apomorphine, chemically modified equivalents which include a pro-drug and pharmaceutical salts thereof.

Another preferred embodiment of the present invention provides for a method of treating sexual dysfunction in a mammal in need of such treatment, including treating impotence; male and female erectile dysfunction; and male and female sexual dysfunction in a human. Particularly, with respect to female sexual dysfunction, the present invention provides a method to increase sexual desire, increase sexual arousal, reduce dyspareunia and/or decrease difficulty in achieving, or inability in achieving, orgasm.

This method includes administering into a nasal cavity of a mammal for absorption through the nasal mucosa thereof a therapeutically effective dosage of a dopamine receptor agonist as previously set forth in combination with a nasal delivery system. The dopamine receptor agonist is preferably selected from a group including apomorphine, chemically modified equivalents which include a pro-drug and pharmaceutical salts thereof. Most preferably a glycol derivative, such as propylene glycol or polyethylene glycol, is included in the method to inhibit and/or eliminate adverse nasal effects. For purposes of the present invention, the nasal delivery system can include a pharmaceutically acceptable buffer, a thickening agent and a humectant.

In another preferred embodiment of the present invention, a method is provided for administering a therapeutically effective amount of a dopamine receptor agonist to a mammal through a nasal membrane without causing substantial intolerable adverse side effects in the mammal. This method includes delivering to a nasal membrane of a mammal a dopamine receptor agonist which is dispersed in a nasal delivery system that includes a pharmaceutically acceptable buffer, a thickening agent and a humectant. In this method, the dopamine receptor agonist is effective for the treatment of a sexual dysfunction in a mammal, including impotence and/or erectile dysfunction in a male mammal, or erectile dysfunction in a female.

Another preferred embodiment of the present invention provides for an intranasal dosage unit for treating sexual dysfunction, including impotency or erectile dysfunction in a mammal, which does not cause substantial intolerable adverse side effects in the mammal. The intranasal dosage unit includes an effective amount of a dopamine receptor agonist in combination with a pharmaceutically acceptable intranasal carrier. This carrier includes a buffer. The pH of the buffer is selected as set forth above to facilitate dopamine receptor agonist absorption through the nasal mucosa so an erection is achieved in about 60 minutes, preferably in about 45 minutes, more preferably in about 30 minutes and most preferably in 15 minutes or less after administration.

The following examples are provided to assist in further understanding the invention. The particular materials and conditions employed are intended to be further illustrative of the invention and are not limiting upon the reasonable scope thereof.

A series of experiments were performed to illustrate features and advantages of the present invention. Several conditions were common to each experiment. For example, in Examples 1-4, apomorphine was administered to healthy male subjects who did not suffer from erectile dysfunction. Further, none of the subjects took any medication for two weeks prior to these experiments.

The following examples show that apomorphine with a glycol derivative can be nasally administered without causing substantial intolerable adverse nasal effects. A different dosage of apomorphine was administered in each experiment to determine a preferred range of dosages having minimal adverse effects.

### EXAMPLES

| Composition of Formulation | Quantity (g/100 g) |
|---|---|
| Apomorphine HCl, USP | 1.00 |
| Citric Acid Anhydrous, USP | 0.67 |
| Sodium Citrate Dihydrate, USP | 0.675 |
| Sodium Metabisulfite, NF | 0.5 |
| Glycerin, USP (96%) | 5.0 |
| Benzalkonium Chloride, NF 50% | 0.04 |
| Purified Water, USP | 100.0 |

Preferred Procedure to Produce Formulation 1

Into a stainless steel beaker equipped with a magnetic stirring bar, add 50% formula weight of Purified Water USP. The formula weight of Citric Acid Anhydrous USP was added and stirred until dissolved. The formula weight of Sodium Citrate Dihydrate USP was added and stirred until dissolved. The formula weight of Sodium Metabisulfate NF was added and stirred until dissolved. The formula weight of Glycerin USP (96%) was added and stirred until dissolved. The formula weight of Benzalkonium Chloride NF (50%) was added and stirred until dissolved. The formula weight of Apomorphine Hydrochloride USP was added and stirred until dissolved. Quantity sufficient the batch with Purified Water USP until 95% of the batch weight is obtained. The pH of the solution can be adjusted with Citric Acid Anhydrous USP or Sodium Citrate Dihydrate USP. Quantity sufficient the batch with Purified Water USP to the required batch size using Purified Water USP. Filter the formulation through a 1.0 micron filter paper.

Another Preferred Procedure to Produce Formulation 1

Into a stainless steel beaker equipped with a magnetic stirring bar, add 90% formula weight of Purified Water USP. The formula weight of Citric Acid Anhydrous USP was added and stirred for 5 minutes. The formula weight of Sodium Citrate Dihydrate USP was added and stirred for 5 minutes. The formula weight of Sodium Metabisulfate NF was added and stirred for 5 minutes. The formula weight of Glycerin USP (96%) was added and stirred for 5 minutes. The formula weight of Benzalkonium Chloride NF (50%) was added and stirred for 5 minutes. The formula weight of Apomorphine Hydrochloride USP was added and stirred for 5 minutes. Quantity sufficient the formulation with Purified Water USP until 95% of the batch weight is obtained. The pH can be adjusted with Citric Acid Anhydrous USP or Sodium Citrate Dihydrate USP. Quantity sufficient the batch with Purified Water USP to the required batch size using Purified Water USP and stir solution for 10 minutes. Filter the formulation through a 1.0 micron filter paper.

### EXAMPLE 1

TABLE I shows the results of an experiment in which 0.5 mg of apomorphine HCI was nasally administered to three male subjects.

TABLE 1

| | NUMBER OF SUBJECTS EXPERIENCING SYMPTOMS | | | | | |
|---|---|---|---|---|---|---|
| | GIVEN BY THE DEGREE OF SEVERITY OF THE SYMPTOM | | | | | |
| | (0.5 mg dose of Formulation 1) (Total subjects = 3) | | | | | |
| | | | | Moderate, | Severe, | |
| | Mild, not | Mild to | Moderate, | somewhat | extremely | TOTAL NUMBER |
| | troublesome, or | moderate, not | somewhat | severe, | troublesome, | OF SUBJECTS |
| SYMPTOMS | of very short | troublesome, of | troublesome, or | troublesome, or | or constantly | EXPERIENCING |
| EXPERIENCED | duration | short duration | persistent | persistent | present | SYMPTOMS |
| Nasal Burning | 2 | 0 | 0 | 0 | 0 | 2 |
| Sneezing | 0 | 0 | 0 | 0 | 0 | 0 |
| Unusual Taste | 1 | 0 | 0 | 0 | 0 | 1 |
| Congestion | 0 | 0 | 0 | 0 | 0 | 0 |
| Smell/Odor | 0 | 0 | 0 | 0 | 0 | 0 |
| Nasal Itching | 0 | 0 | 0 | 0 | 0 | 0 |
| Nasal Pain | 1 | 0 | 0 | 0 | 0 | 1 |
| Total Number of | 4 | 0 | 0 | 0 | 0 | |
| Adverse Nasal | | | | | | |
| Experiences | | | | | | |

One of the subjects of this experiment did not suffer any symptoms and two of the three subjects in this experiment suffered from mild nasal burning or pain. The results of this experiment show some adverse nasal effects with nasal administration of 0.5 mg of apomorphine HCI which does not contain glycol derivatives.

### EXAMPLE 2

TABLE 2 shows the results of an experiment in which 1.0 mg of apomorphine HCI (Formulation 1) was nasally administered to nine male subjects.

TABLE 2

| | NUMBER OF SUBJECTS EXPERIENCING SYMPTOMS | | | | | |
|---|---|---|---|---|---|---|
| | GIVEN BY THE DEGREE OF SEVERITY OF THE SYMPTOM | | | | | |
| | (1.0 mg dose of Formulation 1) (Total subjects = 12) | | | | | |
| | | | | Moderate, | Severe, | TOTAL |
| | Mild, not | Mild to | Moderate, | somewhat | extremely | NUMBER |
| | troublesome, | moderate, not | somewhat | severe, | troublesome, | OF SUBJECTS |
| SYMPTOMS | or of very | troublesome, of | troublesome, | troublesome, | or constantly | EXPERIENCING |
| EXPERIENCED | short duration | short duration | or persistent | or persistent | present | SYMPTOMS |
| Nasal Burning | 2 | 2 | 3 | 0 | 0 | 7 |
| Sneezing | 1 | 0 | 1 | 0 | 0 | 2 |
| Unusual Taste | 3 | 0 | 0 | 0 | 0 | 3 |
| Congestion | 1 | 0 | 0 | 0 | 1 | 2 |
| Smell/Odor | 0 | 0 | 0 | 0 | 0 | 0 |
| Nasal Itching | 0 | 1 | 0 | 0 | 0 | 1 |
| Nasal Pain | 0 | 0 | 0 | 0 | 0 | 0 |
| Total Number of | 7 | 3 | 4 | 0 | 1 | |
| Adverse Nasal | | | | | | |
| Experiences | | | | | | |

It should be noted that some of the subjects of this experiment suffered from more than one symptom and one subject did not suffer from any symptoms.

As shown in TABLE 2, the symptoms suffered by one subject was moderate sneezing; one subject suffered from mild nasal burning; two subjects suffered from mild to moderate nasal burning; one subject from mild to moderate nasal itching; and three subjects from moderate, somewhat troublesome nasal burning.

The results of this experiment show some adverse nasal effects with nasal administration of a 1.0 mg dose apomorphine MCI which does not contain glycol derivatives.

### EXAMPLE 3

TABLE 3 shows the results of an experiment in which 2.0 mg of apomorphine HCI was nasally administered to nine male subjects.

TABLE 3

| | NUMBER OF SUBJECTS EXPERIENCING SYMPTOMS | | | | | |
|---|---|---|---|---|---|---|
| | GIVEN BY THE DEGREE OF SEVERITY OF THE SYMPTOM | | | | | |
| | (2.0 mg dose of Formulation 1) (Total Subjects = 9) | | | | | |
| | Mild, not | | Moderate, | somewhat | extremely | TOTAL NUMBER |
| | troublesome, or | Mild to moderate, | somewhat | severe, | troublesome, | OF SUBJECTS |
| SYMPTOMS | of very short | not troublesome, | troublesome, | troublesome, | or constantly | EXPERIENCING |
| EXPERIENCED | duration | of short duration | or persistent | or persistent | present | SYMPTOMS |
| Nasal Burning | 1 | 0 | 0 | 0 | 0 | 1 |
| Sneezing | 0 | 1 | 0 | 0 | 0 | 1 |
| Unusual Taste | 4 | 0 | 0 | 0 | 0 | 4 |
| Congestion | 0 | 0 | 0 | 0 | 0 | 0 |
| Smell/Odor | 0 | 0 | 0 | 0 | 0 | 0 |
| Nasal Itching | 0 | 0 | 0 | 0 | 0 | 0 |
| Nasal Pain | 0 | 0 | 0 | 0 | 0 | 0 |
| Total Number of | 5 | 1 | 0 | 0 | 0 | |
| Adverse Number | | | | | | |
| Nasal Experiences | | | | | | |

It should be noted that some of the subjects of this experiment suffered from more than one symptom and one subject did not suffer from any symptoms.

One of the nine subjects in this experiment suffered from mild nasal burning; one from mild to moderate nasal burning; and four from mild unusual taste as shown in TABLE 3. The results of this experiment show that there were some adverse nasal effects with the nasal administration of 2.0 mg of apomorphine with HCI without glycol derivatives.

### EXAMPLE 4

TABLE 4 shows the results of an experiment in which 4.0 mg of apomorphine HCI was nasally administered to two male subjects.

TABLE 4

| | NUMBER OF SUBJECTS EXPERIENCING SYMPTOMS | | | | | |
|---|---|---|---|---|---|---|
| | GIVEN BY THE DEGREE OF SEVERITY OF THE SYMPTOM | | | | | |
| | (4.0 mg dose of Formulation 1) (Total Subjects = 2) | | | | | |
| | | Mild to | | Moderate, | Severe, | TOTAL |
| | Mild, not | moderate, not | Moderate, | somewhat | extremely | NUMBER OF |
| | troublesome, | troublesome, | Somewhat | severe, | troublesome, | SUBJECTS |
| SYMPTOMS | or or very | of short | Troublesome, | troublesome, | or constantly | EXPERIENCING |
| EXPERIENCED | short duration | duration | Or persistent | or persistent | present | SYMPTOMS |
| Nasal Burning | 0 | 0 | 0 | 1 | 0 | 1 |
| Sneezing | 0 | 0 | 0 | 0 | 0 | 0 |
| Unusual Taste | 0 | 0 | 0 | 0 | 0 | 0 |
| Congestion | 0 | 0 | 0 | 0 | 0 | 0 |
| Smell/Odor | 0 | 0 | 0 | 0 | 0 | 0 |
| Nasal Itching | 0 | 0 | 0 | 0 | 0 | 0 |
| Nasal Pain | 0 | 0 | 0 | 0 | 0 | 0 |
| Total Number of | 0 | 0 | 0 | 1 | 0 | 0 |
| Adverse Nasal | | | | | | |
| Experiences | | | | | | |

One subject of this experiment experienced moderate sneezing, which was somewhat troublesome as shown in Table 4. Thus, the results of this experiment do show some adverse nasal effects with nasal administration of 4.0 mg of apomorphine HCl which does not contain glycol derivatives.

Examples 1-4 show that nasally-administered apomorphine at different dosages may cause adverse nasal effects.

### EXAMPLE 5

Table 5 shows the results of an experiment in which 1.0 mg of apomorphine HCI which includes a glycol derivative (Formulation 2), in particular propylene glycol, was nasally administered to six males subjects.

| Composition of Formulation | Quantity (g/100 g) |
|---|---|
| Apomorphine HCl, USP | 1.01 |
| Citric Acid Anhydrous, USP | 0.68 |
| Sodium Citrate Dihydrate, USP | 0.44 |
| Propylene Glycol, USP | 7.0 |
| Glycerin, USP (96%) | 4.98 |
| L-Ascorbic Acid, USP | 0.012 |
| Sodium Metabisulfite, NF | 0.088 |
| Edetate Disodium, USP | 0.020 |
| Benzalkonium Chloride, NF (50%) | 0.040 |
| Sodium Hydroxide, NF (1N) | TAP |
| Hydrochloric Acid, NF (1N) | TAP |
| Purified Water, USP | 85.73 |

Preferred Procedure to Produce Formulation 2

Into a stainless steel beaker equipped with a magnetic stirring bar, add 75% formula weight of Purified Water USP. The formula weight of Citric Acid Anhydrous USP was added and stirred for 5 minutes. The formula weight of Sodium Citrate Dihydrate USP was added and stirred for 5 minutes. The formula weight of L-Ascorbic Acid USP was added and stirred for 5 minutes. The formula weight of Sodium Metabisulfate USP was added and stirred for 5 minutes. The formula weight of Edetate Disodium USP was added and stirred for 5 minutes. The formula weight of Benzalkonium Chloride NF was added and stirred for 5 minutes. The formula weight of Glycerin (96%) USP was added and stirred for 5 minutes. The formula weight of Propylene Glycol USP was added and stirred for 5 minutes. The formula weight of Apomorphine Hydrochloride USP was added and stirred for 10 minutes. The pH can be adjusted with Sodium Hydroxide NF (IN) or Hydrochloric Acid NF (IN). Quantity sufficient the solution to final required batch size with Purified Water USP and stir solution for 10 minutes. The pH of the solution can be adjusted with Sodium Hydroxide NF (IN) or Hydrochloric Acid NF (IN). Filter the formulation through a Whatman 4 filter paper and fill the solution in 5 ml amber glass bottles.

Another Preferred Procedure to Produce Formulation 2

Into a stainless steel beaker equipped with a magnetic stirring bar, add 75% formula weight for Purified Water USP. The formula weight of Citric Acid Anhydrous USP was added and stirred for 5 minutes. The formula weight of Sodium Citrate Dihydrate USP was added and stirred for 5 minutes. The formula weight of L-Ascorbic Acid USP was added and stirred for 5 minutes. The formula weight Sodium Metabisulfite USP was added and stirred for 5 minutes. The formula weight of Edetate Disodium USP was added and stirred for 5 minutes. The formula weight of Benzalkonium Chloride NF was added and stirred for 5 minutes. The formula weight of Glycerin (96%) USP was added and stirred for 5 minutes. The formula weight of Propylene Glycol USP was added and stirred for 5 minutes. The formula weight of Apomorphine Hydrochloride USP was added and stirred for 10 minutes. The pH can be adjusted with Sodium Hydroxide NF (IN) or Hydrochloric Acid NF (IN). Quantity sufficient the solution to final required batch size with Purified Water USP and stir solution for 10 minutes. The pH can be adjusted with Sodium Hydroxide NF (IN) or Hydrochloric Acid NF (IN). Filter the formulation through a Whatman 4 filter paper and fill the solution in 5ml amber glass bottles.

TABLE 5

| | NUMBER OF SUBJECTS EXPERIENCING SYMPTOMS | | | | | |
|---|---|---|---|---|---|---|
| | GIVEN BY THE DEGREE SEVERITY OF THE SYMPTOM | | | | | |
| | (1.0 mg dose of Formulation 2) (Total Subjects = 6) | | | | | |
| | | | | Moderate, | Severe, | TOTAL |
| | | | Moderate, | somewhat | extremely | NUMBER |
| | Mild, not | Mild to moderate, | somewhat | severe, | troublesome, | OF SUBJECTS |
| SYMPTOMS | troublesome, or of | not troublesome, | troublesome, or | troublesome, | or constantly | EXPERIENCING |
| EXPERIENCED | very short duration | of short duration | persistent | or persistent | present | SYMPTOMS |
| Nasal Burning | 0 | 0 | 0 | 0 | 0 | 0 |
| Sneezing | 0 | 0 | 0 | 0 | 0 | 0 |
| Unusual Taste | 0 | 0 | 0 | 0 | 0 | 0 |
| Congestion | 0 | 0 | 0 | 0 | 0 | 0 |
| Smell/Odor | 0 | 0 | 0 | 0 | 0 | 0 |
| Nasal Itching | 0 | 0 | 0 | 0 | 0 | 0 |
| Nasal Pain | 0 | 0 | 0 | 0 | 0 | 0 |
| Total Number of | 0 | 0 | 0 | 0 | 0 | 0 |
| Adverse Nasal | | | | | | |
| Experiences | | | | | | |

As shown in Table 5, none of the subjects suffered from adverse nasal effects. The results of this experiment show that a glycol derivative, in particular propylene glycol, inhibited/eliminated the adverse nasal effects when added to a nasally administered apomorphine composition.

### EXAMPLE 6

Table 6 shows the results of an experiment in which 2.0 mg of apomorphine HCl which includes a glycol derivative, in particular propylene glycol, (Formulation 2) was nasally administered to six male subjects.

TABLE 6

| | NUMBER OF SUBJECTS EXPERIENCING SYMPTOMS | | | | | |
|---|---|---|---|---|---|---|
| | GIVEN BY THE DEGREE SEVERITY OF THE SYMPTOM | | | | | |
| | (2.0 mg dose of Formulation 2) (Total Subjects = 6) | | | | | |
| | | Mild to | | Moderate, | Severe, | |
| | Mild, not | moderate, not | Moderate, | somewhat | extremely | TOTAL NUMBER |
| | troublesome, | troublesome, | somewhat | severe, | troublesome, | OF SUBJECTS |
| SYMPTOMS | or of very | of short | troublesome, | troublesome, | or constantly | EXPERIENCING |
| EXPERIENCED | short duration | duration | or persistent | or persistent | present | SYMPTOMS |
| Nasal Burning | 0 | 0 | 0 | 0 | 0 | 0 |
| Sneezing | 0 | 0 | 0 | 0 | 0 | 0 |
| Unusual Taste | 1 | 0 | 0 | 0 | 0 | 1 |
| Congestion | 0 | 0 | 0 | 0 | 0 | 0 |
| Smell/Odor | 0 | 0 | 0 | 0 | 0 | 0 |
| Nasal Itching | 0 | 0 | 0 | 0 | 0 | 0 |
| Nasal Pain | 0 | 0 | 0 | 0 | 0 | 0 |
| Total Number of | 1 | 0 | 0 | 0 | 0 | |
| Adverse | | | | | | |
| Experiences | | | | | | |

As shown in Table 6 none of the subjects suffered from adverse nasal effects. The results of this experiment show that a glycol derivative, in particular propylene glycol, inhibited/eliminated the adverse nasal effects when added to a nasally administered apomorphine composition.

Thus, while there have been described what are presently believed to be the preferred embodiments of the present invention, those skilled in the art will realize that other and further embodiments can be made without departing from the spirit and scope of the invention, and it is intended to include all such further modifications and changes as come within the true scope of the claims set forth herein.

TABLE 9
Attributes of the in-clinic efficacy and safety study conducted
on nasal apomorphine HCl in patients suffering from psychogenic
erectile dysfunction. Using Formulation 2.
Formulation 2 includes propylene glycol.

| # | Parameter | Explanation |
|---|---|---|
| 1 | Patient population | Heterosexual males suffering from psychogenic |
| | | erectile dysfunction |
| 2 | Primary inclusion | Responder to Caverject (alprostadil product) |
| | criterion | within 1 yr of the study |
| 3 | Patient age range | 18-55 |
| 4 | Study type | Double blind double dummy placebo controlled |
| | | cross over |
| 5 | Positive control | Viagra 50 mg tablets placed in colored capsules |
| | | of adequate size |
| 6 | Type of sexual | Sexually explicit video tapes over a 90 |
| | stimulation | minute period |
| 7 | Patient interaction | Patients were undisturbed during the 90 |
| | | minute study period post dosing |
| 8 | Type of | Private rooms on a hospital floor |
| | environment | |
| 9 | Nasal placebo | Matching nasal formulation |
| 10 | Oral placebo | Color capsules, same as used in the Viagra |
| | | group |
| 11 | Nasal Apomorphine | 0.25 mg; 0.50 mg; 1.0 mg |
| | HCl doses | |
| 12 | Number and volume | One spray of 0.1 ml in one nostril |
| | of nasal spray | |
| 13 | Criteria of efficacy | Self graded questionnaire |
| | evaluation | |
| 14 | Criteria of safety | Vitals pre-dosing, self assessment during 90 |
| | assessment | min study followed by vitals |
| 15 | Blood sampling for | No blood samples were withdrawn |
| | absorption | |

TABLE 10
Overall summary of results from an in-clinic efficacy and safety study conducted on nasal apomorphine
HCl (Formulation 2) as a function of dose against matching nasal placebo and 50 mg Viagra tablets.
Double blind, double dummy, placebo controlled crossover study against a positive control.

| | | | | Results ± Standard Deviation | | | | | | | | | |
|---|---|---|---|---|---|---|---|---|---|---|---|---|---|
| | | | | Erection | | | | | | Global Assess | | | |
| | | Dose | | | | | | Satisfaction | | Score * | | | |
| # | Product | (mg) | n | YES | NO | Onset Min | Duration Min | Yes | No | 1 | 2 | 3 | 4 |
| 1 | Nasal placebo | — | 20 | 18 | 2 | 26.8 ± 13.3 | 15.1 ± 9.20 | 6 | 11 | 4 | 7 | 5 | 2 |
| 2 | Viagra | 50 | 21 | 15 | 6 | 21.7 ± 12.2 | 22.8 ± 15.5 | 7 | 8 | 2 | 3 | 6 | 4 |
| 3 | Nasal Apo | 0.25 | 19 | 15 | 4 | 21.4 ± 8.60 | 20.5 ± 12.1 | 12 | 3 | 1 | 1 | 6 | 7 |
| 4 | Nasal Apo | 0.50 | 19 | 17 | 2 | 22.7 ± 12.7 | 19.5 ± 11.3 | 14 | 3 | 1 | 2 | 7 | 7 |
| 5 | Nasal Apo | 1.00 | 21 | 18 | 3 | 21.7 ± 13.5 | 221.8 ± 12.5 | 13 | 5 | 2 | 4 | 6 | 6 |

* Self Grading of Penile Erection:
1 = Increase in size but not hard
2 = Hard, but not hard enough for vaginal penetration
3 = Hard enough for vaginal penetration (but not completely hard)
4 = Completely hard

The results of this experiment show that nasal administration of apomorphine HCI containing propylene glycol was effective in inducing an erection. Global assessment scores of 3 and 4 indicate erections which were sufficient for vaginal penetration. Also the nasal administration of apomorphine HCI containing propylene glycol was more effective than a nasal placebo or Viagra in inducing an erection.

TABLE 11
Summary of the Efficacy Assessment of in-clinic study for nasal
apomorphine HCl (Formulation 2) as a function of dose, against
matching nasal placebo and 50 mg Viagra tablets. Double blind,
double dummy, placebo controlled crossover study against a
positive control.

| | | | | % Subjective | | % Erection Assessment | | | | |
|---|---|---|---|---|---|---|---|---|---|---|
| | | Dose | | Erection Quality | | Based on Global Score * | | | | |
| # | Product | (mg) | n | Yes | Satisfactory | 1 | 2 | 3 | 4 | (3 + 4) |
| 1 | Nasal placebo | — | 20 | 90.0 | 30.0 | 20.0 | 35.0 | 25.0 | 10.0 | 35.0 |
| 2 | Viagra | 50 | 21 | 71.4 | 33.3 | 9.5 | 14.3 | 28.6 | 19.0 | 47.6 |
| 3 | Nasal Apo | 0.25 | 19 | 78.9 | 63.2 | 5.3 | 5.3 | 31.7 | 36.8 | 68.5 |
| 4 | Nasal Apo | 0.50 | 19 | 89.5 | 73.7 | 5.3 | 10.5 | 36.8 | 36.8 | 73.6 |
| 5 | Nasal Apo | 1.00 | 21 | 85.7 | 61.9 | 9.5 | 19.0 | 28.6 | 28.6 | 57.2 |

* Self Grading of Penile Erection:
1 = Increase in size but not hard
2 = Hard, but not hard enough for vaginal penetration
3 = Hard enough for vaginal penetration (but not completely hard)
4 = Completely hard

The results of this experiment show that nasal administration of apomorphine HCl containing propylene glycol was effective in inducing an erection. Also the nasal administration of apomorphine HCl was more effective than nasal placebo or Viagra in inducing an erection.

We claim:

1. A nasally administered pharmaceutical composition for treating sexual dysfunction in a mammal comprising a therapeutically effective amount of a dopamine receptor agonist in combination with a glycol derivative wherein incidence of adverse nasal effects of said mammal is reduced.

2. The nasally administered pharmaceutical composition of claim 1, wherein said dopamine receptor agonist is selected from the group consisting of apomorphine, chemically modified equivalents and pharmaceutical solids thereof.

3. The nasally administered pharmaceutical composition of claim 1, wherein said glycol derivative is propylene glycol.

4. The nasally administered pharmaceutical composition of claim 1, wherein said glycol derivative is polyethylene glycol.

5. The nasally administered pharmaceutical composition of claim 1, wherein said composition further comprises glycerin.

6. A nasally administered pharmaceutical composition for treating sexual dysfunction in a mammal comprising a therapeutically effective amount of a dopamine receptor agonist in combination with propylene glycol.

7. The nasally administered pharmaceutical composition of claim 6, wherein said dopamine receptor agonist is selected from a group of apomorphine, chemically modified equivalents of pharmaceutical solids thereof.

8. A method of treating sexual dysfunction in a mammal comprising nasally administering a therapeutically effective amount of a dopamine receptor agonist in combination with a glycol derivative before, during or after sexual activity, wherein incidence of adverse nasal effects of said mammal is reduced.

9. The method of claim 8, wherein said dopamine receptor agonist is selected from the group consisting of apomorphine, chemically modified equivalents and pharmaceutical solids thereof.

10. A method of increasing sexual desire of a female or male mammal comprising nasally administering a therapeutically effective amount of a dopamine receptor agonist in combination with a glycol derivative before, during or after sexual activity wherein incidence of adverse nasal effects of said mammal are reduced to said mammal.

11. The method according to claim 10, wherein said dopamine receptor agonist is selected. from the group consisting of apomorphine, chemically modified equivalents and pharmaceutical solids thereof.

12. A method of increasing sexual arousal in a female or male mammal comprising nasally administering a therapeutically effective amount of a dopamine receptor agonist to said mammal before, during or after sexual activity.

13. The method according to claim 12 further comprising a glycol derivative wherein incidence of adverse nasal effects of said mammal are reduced.

14. The method according to claim 12 wherein said dopamine receptor agonist is selected from the group consisting of apomorphine, chemically modified equivalents and pharmaceutical solids thereof.

15. A method of reducing dyspareumia in a female mammal comprising nasally administering a therapeutically effective amount of a dopamine receptor agonist to said mammal before, during or after sexual activity.

16. The method according to claim 15 further comprising a glycol derivative wherein incidence of adverse nasal effects of said mammal are reduced.

17. The method according to claim 15 wherein said dopamine receptor agonist is selected from the group consisting of apomorphine, chemically modified equivalents and pharmaceutical solids thereof.

18. A method of reducing difficulty in achieving or inability of achieving orgasm in a female mammal comprising nasally administering a therapeutically effective amount of a dopamine receptor agonist to said mammal before, during or after sexual activity.

19. The method according to claim 18 further comprising a glycol derivative wherein incidence of adverse nasal effects of said mammal are reduced.

20. The method according to claim 18 wherein said dopamine receptor agonist is selected from the group consisting of apomorphine, chemically modified equivalents and pharmaceutical solids thereof.
